(19) United States
(12) Patent Application Publication
(10) Pub. No.: US 2024/0148988 A1
(43) Pub. Date: May 9, 2024

(54) SOLENOIDS FOR INSUFFLATION SYSTEMS

(71) Applicant: Conmed Corporation, Largo, FL (US)

(72) Inventor: Michael L. Koltz, Jr., Aurora, CO (US)

(73) Assignee: Conmed Corporation, Largo, FL (US)

(21) Appl. No.: 18/279,977

(22) PCT Filed: Mar. 7, 2022
(86) PCT No.: PCT/US2022/019073

§ 371 Date: Sep. 1, 2023

Related U.S. Application Data
(60) Provisional application No. 63/158,090, filed on Mar. 8, 2021.

(51) Int. Cl. A61M 13/00; A61B 17/34

(52) U.S. Cl. CPC A61M 13/003; A61B 17/3474

(57) ABSTRACT

A method includes applying an actuating voltage to a solenoid for a first time span to actuate the solenoid against a bias. The method includes lowering voltage applied to the solenoid to a holding voltage that is lower than the actuating voltage to hold the solenoid against the bias for a second span of time. The solenoid has a coil to which the actuating and holding voltages are applied. The coil is rated for continuous operation at a rated voltage. The actuating voltage is above the rated voltage.

### CROSS-REFERENCE TO RELATED APPLICATIONS

This application claims benefit of priority to U.S. Provisional Patent Application No. 63/158,090 filed Mar. 8, 2021 the content of which is incorporated by reference herein in its entirety.

### BACKGROUND

### 1. Field

The present disclosure relates to solenoids, and more particularly to solenoid valves such as used in insufflation systems.

### 2. Description of Related Art

Insufflators and gas sealed trocar systems commonly employ solenoids to control gas flow. These solenoids inherently produce thermal energy when electrically energized due to the electrical resistance of the coil wire. This heat must be dissipated, commonly by use of cooling fans, which further burdens the system with additional electrical power demands, increased size, and increased weight. Modern operating rooms are continually challenged by an increasing amount of medical electrical equipment being used for surgical procedures, and this equipment consumes space, electrical power, and presents musculoskeletal injury hazards to personnel who must constantly lift and circulate equipment between procedures. Therefore, there persists a continual need for smaller, lighter, and more electrically efficient medical devices.

The solenoids used within insufflator and gas sealed trocar systems, typically numbering between three and nine, contribute substantially to systems overall size, weight, and power consumption. Solenoid size increases with increasing pressure and flow rate requirements. This phenomenon is related to the increased solenoid force required to displace the poppet as the pressure or pressure area acting on the poppet increases.

The conventional techniques have been considered satisfactory for their intended purpose. However, there is an ever present need for improved systems and methods for smaller, lighter, and more efficient solenoids and solenoid valves. This disclosure provides a solution for this need.

### SUMMARY

A method includes applying an actuating voltage to a solenoid for a first time span to actuate the solenoid against a bias. The method includes lowering voltage applied to the solenoid to a holding voltage that is lower than the actuating voltage to hold the solenoid against the bias for a second span of time. The solenoid has a coil to which the actuating and holding voltages are applied. The coil is rated for continuous operation at a rated voltage. The actuating voltage is above the rated voltage.

The holding voltage can be below the rated voltage. Applying the actuating voltage to the solenoid can be performed after the bias is relaxed and voltage applied to the solenoid is dropped below the holding voltage for a span of time during which the coil has cooled to a first temperature. The coil can be raised to a second temperature during the second span of time. The first time span can be short enough so the coil does not exceed the second temperature during the first span of time. The actuating voltage can be higher than the rated voltage, but the first span of time can be short enough to apply the first voltage without causing a failure mode for the coil wherein the failure mode includes at least one of insulation breakdown, housing or bobbin melting or distortion, or armature seizing. At least one of the actuating voltage and the holding voltage can be applied as an alternating waveform that averages at the respective actuating and/or holding voltage.

The solenoid can be operatively connected to a valve having an open position for allowing flow through the valve and a closed position for preventing flow through the valve. The bias can bias the valve to the open position. Actuating the solenoid against the bias can actuate the valve to the closed position. Holding the solenoid against the bias can hold the valve in the closed position. The method can include lowering voltage below the holding voltage in response to over pressure in an a insufflation line, thereby opening the valve to the open position under the bias to relieve the over pressure in the insufflation line. It is also contemplated that the method can include lowering voltage to zero in a loss of power event, thereby opening the valve to the open position under the bias to relieve pressure in a surgical cavity in a patient. The method can include filtering flow through the valve in the open position to reduce or prevent particulates passing through the valve.

The actuating voltage can be 48 Volts and the holding voltage can be 5 Volts, for example. Applying the actuating voltage can include applying 20 Watts to the solenoid, wherein holding the solenoid against the bias for a second span of time includes applying 1 Watt to the solenoid at the holding voltage, for example.

A solenoid includes a coil seated in a housing. An armature extends in a longitudinal direction through the coil and out of a first end of the housing. The armature includes a material responsive to external magnetic fields and is mounted in the hosing for movement relative to the coil and housing responsive to a magnetic field of the coil. A latching member is mounted at a second end of the housing and including a latching surface configured to contact the armature with the armature in a retracted position. A plurality of permanent magnets circumferentially distributed around the armature at the first end of the housing.

The latching member can be engaged with threads to the housing for adjusting axial position of the latching surface within the housing by turning the latching member relative to the housing. There can be four permanent magnets circumferentially distributed about the armature. The polarity of each of the permanent magnets can be oriented radially relative to the longitudinal direction. For each of the permanent magnets, a radially inner pole can be magnetic south and a radially outer pole can be magnetic north. The coil can be wound around an outward surface of a bobbin of the housing, wherein an inward facing surface of the bobbin engages the armature as a sliding bearing surface.

A system includes a solenoid having a coil seated in a housing. An armature extends in a longitudinal direction through the coil and out of a first end of the housing. The armature includes a material responsive to external magnetic fields and is mounted in the hosing for movement relative to the coil and housing responsive to a magnetic field of the coil. A controller is operatively connected to control the coil. The controller includes machine readable instructions configured to cause the controller to apply an actuating voltage to the coil for a first time span to actuate the solenoid against a bias and lower voltage applied to the solenoid to a holding voltage that is lower than the actuating voltage to hold the solenoid against the bias for a second span of time, wherein the coil is rated for continuous operation at a rated voltage, and wherein the actuating voltage is above the rated voltage.

A valve member can be mounted for movement together with the armature. A valve housing can define a flow path from an inlet of the valve housing to an outlet of the valve housing. The flow path can pass through a valve seat. With the valve member and armature in a first position, the valve member can seal against the valve seat preventing flow through the flow path. With the valve member and armature in a second position, the valve member can be spaced apart from the valve seat, allowing flow through the flow path. A filter medium can be seated in the flow path in the valve housing downstream of the valve seat, configured to prevent particle flow out of the valve housing with the armature and valve member in the second position.

An insufflator can include a pressure source. A pneumatic line can connect the pressure source to a connector configured to connect the insufflator to a trocar tube set for insufflating a surgical cavity of a patient. The inlet of the valve housing can connected to a branch off of the pneumatic line to selectively allow or block pressure relief flow from the pneumatic line.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 is a schematic perspective view of an embodiment of a system constructed in accordance with the present disclosure, showing an insufflator providing insufflation to a patient;

FIG. 2 is a schematic view of the system of FIG. 1, showing the solenoid and valve connected to a branch in a gas line that runs between the pressure source and the trocar;

FIG. 3 is a cross-sectional side elevation view of the solenoid and valve of FIG. 2, showing the valve in the closed or no-flow through position;

FIG. 4 is a cross-sectional side elevation view of the solenoid and valve of FIG. 3, showing the valve in the open position allowing flow through the valve;

FIG. 5 is a cross-sectional plan view of the solenoid of FIG. 3, showing the permanent magnets;

FIG. 6 is a function block diagram for the system of FIG. 2;

FIG. 7 is a diagram of voltage and state effects for the system of FIG. 2; and

FIG. 8 is a graph of voltage versus time showing examples of non-periodic valve cycling for the system of FIG. 2.

### DETAILED DESCRIPTION OF THE PREFERRED EMBODIMENTS

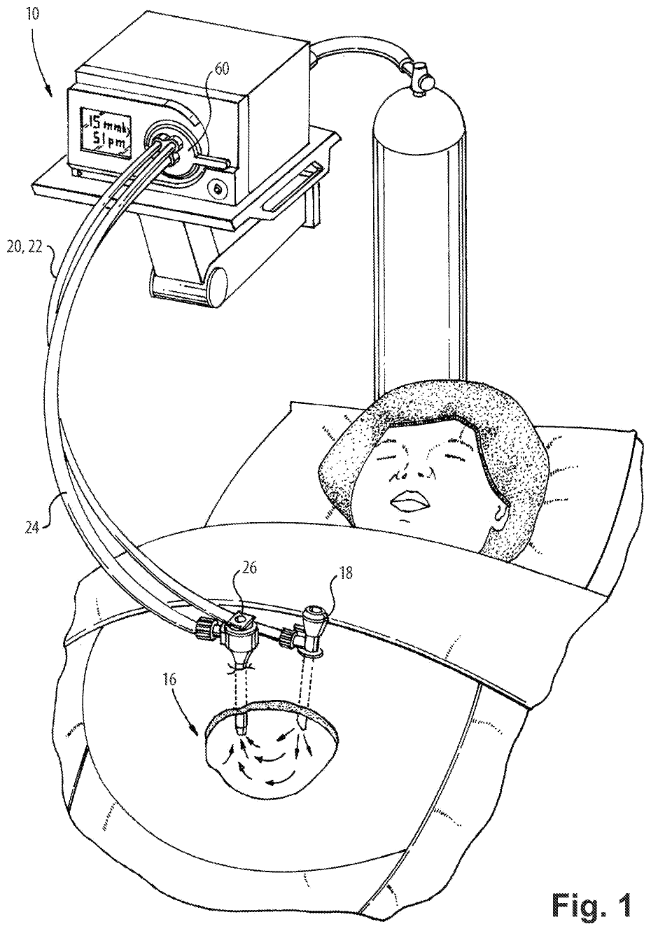

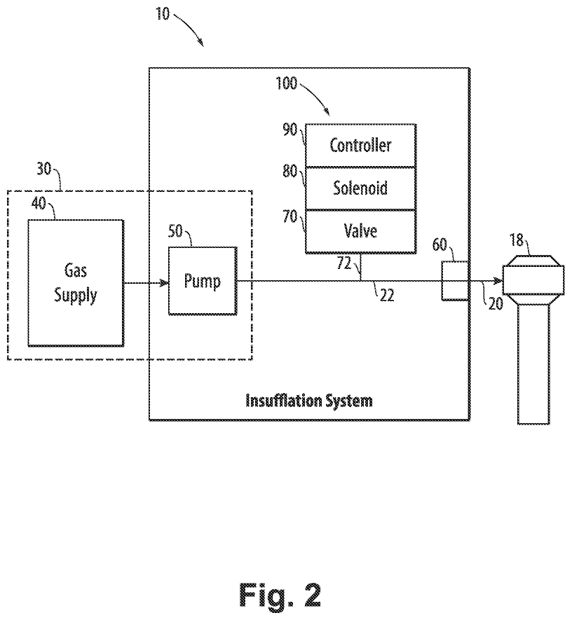

Reference will now be made to the drawings wherein like reference numerals identify similar structural features or aspects of the subject disclosure. For purposes of explanation and illustration, and not limitation, a partial view of an embodiment of a solenoid vale system in accordance with the disclosure is shown in FIG. 2 and is designated generally by reference character 100. Other embodiments of systems in accordance with the disclosure, or aspects thereof, are provided in FIGS. 1, and 3-8, as will be described. The systems and methods described herein can be used to actuate valves such as in insufflators, wherein the respective solenoids have improved size, weight, and efficiency than in more traditional solenoid valves.

Referring now to FIG. 1, there is illustrated an insufflator or insufflation system 10. The insufflator 10 includes an inlet flow path 22 leading to a first trocar 18 communicating with the surgical cavity 16 of a patient, through which a flow of insufflation gas is delivered to the surgical cavity 16. The insufflator 10 optionally includes an outlet flow path 24 leading from a second trocar 26 communicating with the surgical cavity 16, though which a flow of smoky gas can be removed from the surgical cavity 16, for example. While shown and described herein in the exemplary context of mechanically sealed trocars, those skilled in the art will readily appreciate that systems and methods as disclosed herein can be used with pneumatically sealed and/or mechanically sealed trocars without departing from the scope of this disclosure.

With reference now to FIG. 2, the insufflator 10 includes a pressure source 30, which can include an external pressurized gas supply 40 connected to the insufflator 10, and/or a pump 50, which can maintain insufflation pressure, e.g. if pressure in the external gas supply 40 is inadequate on its own for insufflation. A pneumatic line, i.e. the inlet flow path 22, connects the pressure source 30 to a connector 60 configured to connect the insufflator 10 to a trocar tube set 20 for insufflating a surgical cavity 16 of the patient as shown in FIG. 1. A valve 70 is in fluid communication with a branch 72 of the inlet flow path 22 to provide pressure relief to the inlet flow path 22. A solenoid 80 is operatively connected to actuate the valve 70, and a controller 90 is in turn connected to control the solenoid 80 for controlled actuation of the valve 70.

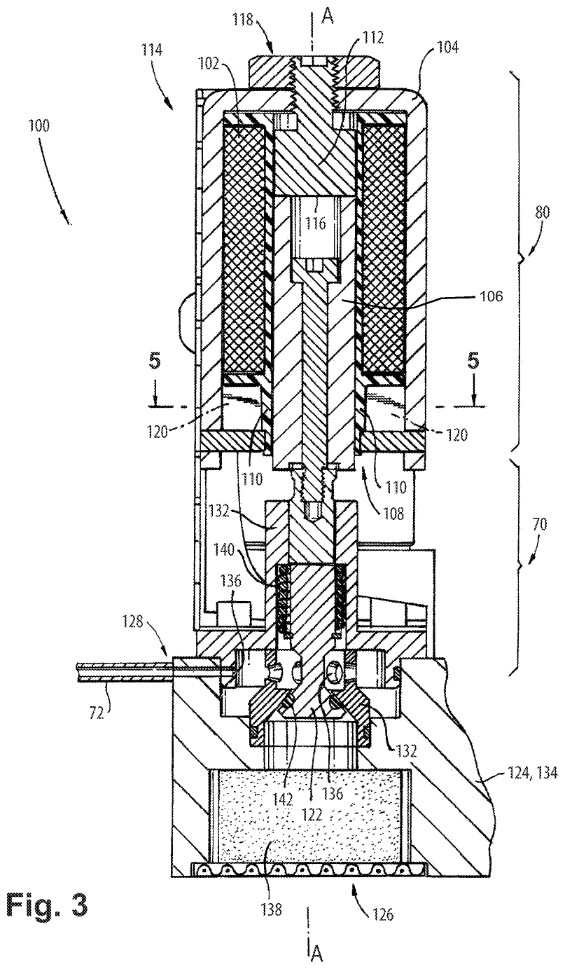

Referring now to FIG. 3, a solenoid valve system 100 is described. The system 100 generally includes the solenoid 80, and the valve 70 that are shown in FIG. 2. The solenoid 80 includes a coil 102 seated in a housing 104 and winding around the longitudinal axis A. An armature, or plunger 106 extends in a longitudinal direction, i.e. along axis A, through the coil 102 and out of a first end 108 of the housing 104. The armature 106 includes a material responsive to external magnetic fields and is mounted in a bobbin 110 of the housing 104 for longitudinal movement relative to the coil 102 and housing 104 responsive to a magnetic field of the coil 102. The coil 102 is wound around an outward surface of the bobbin 110 of the housing 104, wherein an inward facing surface of the bobbin 110 engages the armature 106 as a sliding bearing surface.

With continued reference to FIG. 3, a latching member 112 is mounted at a second end 114 of the housing 104 opposite the first end 108. The latching member 112 includes a latching surface 116 configured to contact the armature 106 with the armature 106 in a retracted position as shown in FIG. 3. The latching member 112 is engaged with threads 118 to the housing 104 for adjusting the axial position (relative to axis A) of the latching surface 116 within the housing 104 by turning the latching member 112 relative to the housing 104.

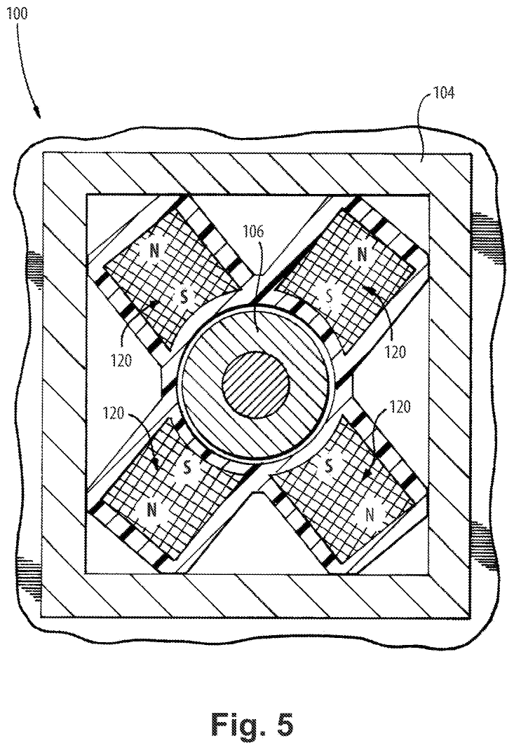

A plurality of permanent magnets 120 are circumferentially distributed around the armature 106 at the first end 108 of the housing 104 for shaping the magnetic field of the coil 102. As shown in the cross-section in FIG. 5, there are four permanent magnets 120 circumferentially distributed about the armature 106 in a symmetric pattern as to not bias the lateral forces on the plunger. The polarity of each of the permanent magnets 120 is oriented radially relative to the longitudinal direction shown in FIG. 3. As shown in FIG. 5, for each of the permanent magnets 120, the radially inner pole is magnetic south (indicated in FIG. 5 as S) and the radially outer pole is magnetic north (indicated in FIG. 5 as N).

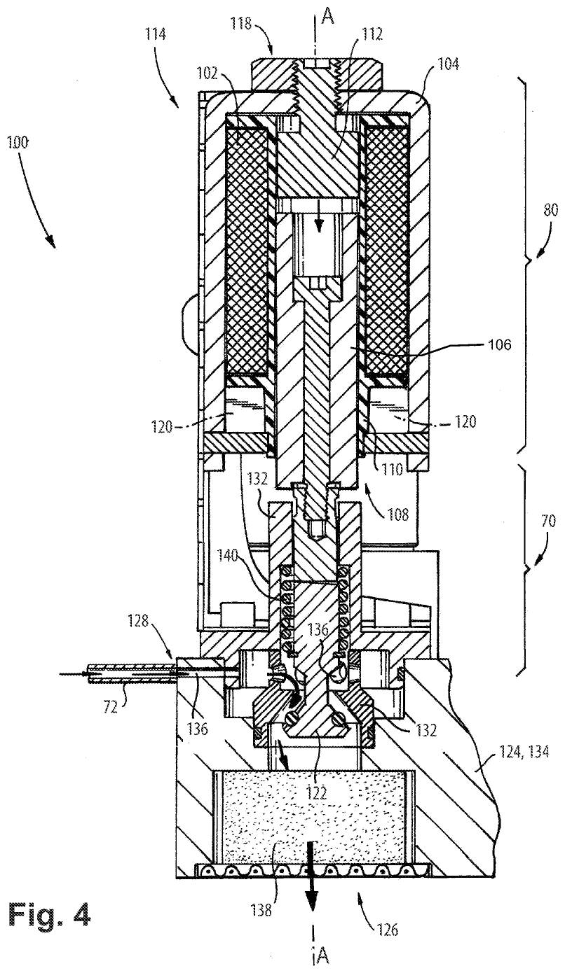

With reference again to FIGS. 3-4, a valve member 122, or plunger, is mounted for movement together with the armature 106. A valve housing 124 defines a flow path from an inlet 128 of the valve housing 124 to an outlet 126 of the valve housing 128. The flow path passes through a valve seat 130 of the valve housing 124, and is indicated in FIG. 4 with the heavy flow arrows. The valve housing 124 includes a bearing member 132, an inside surface of which acts as a sliding bearing surface for linear movement of the valve member 122. The valve seat 130 is positioned between the bearing member 132 and the manifold portion 134 of the valve housing 124. The flow path shown in FIG. 4 includes passages 136, e.g. radial passages or the like, through each of the manifold portion 134, vale seat 130, and bearing member 132.

With the valve member 122 and armature 106 in a first position, or closed position shown in FIG. 3, the valve member 122 seals against the valve seat 130 preventing flow through the flow path. With the valve member and armature in a second position, or open position shown in FIG. 4, the valve member 122 is spaced apart from the valve seat 130, allowing flow through the flow path. A filter medium 138 is seated in the flow path in the outlet 126 of the valve housing 124 downstream of the valve seat 130. The filter medium 138 is configured to prevent particle flow, such as condensation, out of the valve housing 124 into the enclosure of the insufflator 10, e.g. during pressure relief from gases in the surgical cavity 16 of FIG. 1, with the armature 106 and valve member 122 in the second position or open position shown in FIG. 4. The inlet 128 of the valve housing 124 is connected to the branch 72 off of the pneumatic line 22 (labeled in FIGS. 1 and 2) to selectively allow or block pressure relief flow from the pneumatic line 22. When the valve 70 is open, compressed flow is diverted from gas jets through filter medium 138. The flow direction is reversed from that in more traditional systems to enable high pressure to blow open the valve 70 by overcoming the net forces of the spring 140 and solenoid 80 under the hold-open voltage V_{low}.

With continued reference to FIGS. 3-4, a spring 140 is seated between a seat in the bearing member 132 and the valve member 122. The spring 140 biases the valve member 122 and armature 106 toward the open position shown in FIG. 4. So in the absence of sufficient power to the coil 102, the magnetic forces acting on the armature 106 are weaker than the bias of the spring 140, so the valve member 122 moves to the open position shown in FIG. 4. With sufficient power to the coil 102, the magnetic forces acting on the armature 106 can overcome the bias of the spring 140, moving the valve member 122 to the open position shown in FIG. 3. The adjustment of the position of the latching surface 116 of the latching member 112 in the housing 104, as described above, can be used to prevent over compressing a seal element 142 of the valve member 122 in the closed position shown in FIG. 3.

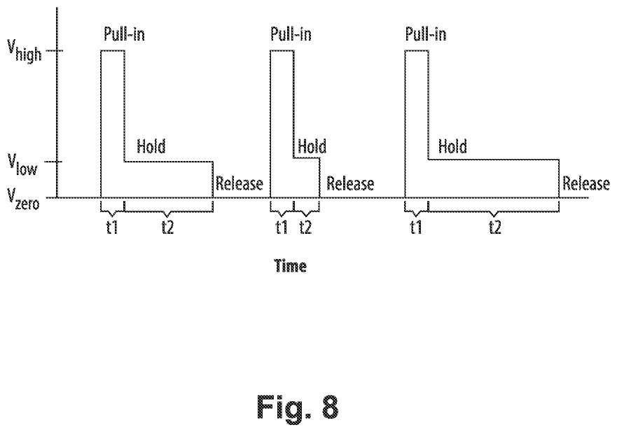

With reference now to FIGS. 3 and 8, the solenoid valve system 100 includes the controller 90 (labeled in FIG. 2), which is operatively connected to control the coil 102 of the solenoid 80. The controller 90 includes machine readable instructions configured to cause the controller 90 to apply an actuating voltage V_{high} to the coil 102 for a first time span t1 to actuate the solenoid 80 (and valve 70) against the bias of the spring 140 and then lower voltage applied to the coil 102 of the solenoid 80 to a holding voltage V_{low} that is lower than the actuating voltage V_{high} to hold the armature 106 of the solenoid 80, as well as the valve member 122, in the open position shown in FIG. 4 against the bias for a second span of time t2. FIG. 8 shows a graph with three examples of the voltage over time cycle described above. The actuating voltage V_{high} can be 48 Volts and the holding voltage V_{low} can be 5 Volts, for example. Applying the actuating voltage V_{high} can include applying 20 Watts to the solenoid, wherein holding the solenoid against the bias for a second span of time includes applying 1 Watt to the solenoid at the holding voltage V_{low}, for example.

The coil 102 of FIGS. 3-4 is rated for continuous operation at a rated voltage. The actuating voltage V_{high} is above the rated voltage. This is possible because the holding voltage V_{low} is below the rated voltage, and because the first span of time t1 is relatively short. The controller 90 (of FIG. 2) applies the actuating voltage to the solenoid 80 after the spring bias is relaxed and voltage applied to the solenoid is dropped below the holding voltage V_{low} for a span of time during which the coil 102 is cooled to a first temperature. The coil 102 is brought to a second temperature during the second span of time t2, e.g., an operation temperature during the holding of the solenoid valve system 100 in the closed position shown in FIG. 3. The first time span t1 at the actuating voltage V_{high} is short enough so the coil 102 does not exceed the second temperature during the first span of time, i.e. the coil 102 is only over its rated voltage long enough to heat up to the coil's safe operating temperature. The actuating voltage V_{high} is higher than the rated voltage, but the first span of time t1 is short enough to apply the first voltage V_{high} without causing a failure mode for the coil 102. Coil failure modes can include any of insulation breakdown, housing or bobbin melting or distortion, or armature seizing. At least one of the actuating voltage V_{high} and the holding voltage V_{low} can be applied as an pulse width modulating (PWM) waveform or alternating waveform that yields and average voltage at the respective actuating and/or holding voltage. Any suitable waveform can be used, such as alternating current waveforms generated by analog methods taking on common forms including but not limited to square, triangular, sinusoidal, sawtooth, and half-sine. Also, the waveform can be irregular or dynamically changing by, for example digital signal generation, so long as the averaged effect yield the voltage. Continuous direct current can also be used.

In the event that the valve 70 is in the closed position, shown in FIG. 3, and the controller 90 detects an over pressure in an the insufflation line 22 (labeled in FIG. 2), the controller 90 can lower the voltage on the coil 102 below the holding voltage V_{low} thereby opening the valve 80 to the open position shown in FIG. 4 under the spring bias to relieve the over pressure in the insufflation line 22. Similarly, if there is a loss of power event that lowers voltage to the coil 102 to zero, the valve 70 will open to the open position shown in FIG. 4 under the spring bias to relieve pressure in a surgical cavity 16 in a patient (labeled in FIG. 1) through the insufflation line 22 and its branch 72, labeled in FIGS. 2-4.

This disclosure describes a solenoid and a solenoid controller employing a control having at least two voltage states, with at least two being non-zero, applied to the solenoid coil. The first state is a voltage higher than the second and is selected to have sufficient pull-in force to actuate the valve. The first voltage is held momentarily until the solenoid plunger has displaced far enough for the second state voltage to hold the plunger in the pulled-in state. The second voltage is selected to maintain the pulled-in state in opposition to return forces including a return spring or gas pressure. Additionally, the second state voltage is selected to reduce the required coil size and heat losses.

Finally, a third voltage state, which may be zero, reduces the holding force to a value less than the sum of the return spring and pressure forces acting on the poppet causing the poppet to return to its position prior to pull-in.

In one embodiment the first voltage is 24 Volts an applied for duration of one second. The second voltage state serving the hold function is 10 Volts, and the third voltage state, serving the release function is zero. In all embodiments, the second voltage can be between 10% and 90% of the first voltage.

The individual voltages may be continuous direct current or, in other embodiments, may be pulse width modulated with periodic averaging yield voltage steps. In yet other embodiments, the voltage steps may not be distinct, but may be a continuously decreasing voltage.

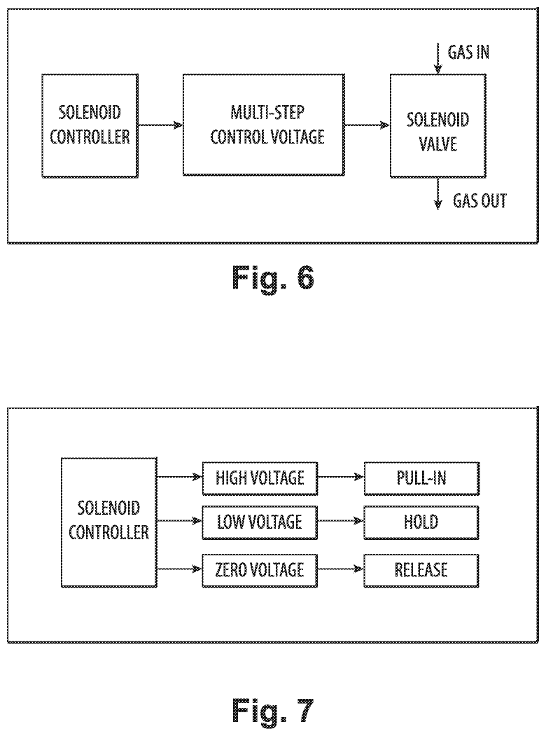

FIG. 6 shows a functional block diagram wherein the solenoid controller provides the multi-step control voltage described above to the solenoid, which has the physical effect of allowing or disallowing gas flow into and out of the valve. FIG. 7 sows the voltage states and effects described above.

Steady-state current limits can be exceeded for short durations, and similarly, duty cycles less than 100% allow steady state current limits to be exceeded. This is allowable due to the time required for the resistive heat generation to overcome the thermal capacity of the coil materials and raise the coil temperature to a damaging level.

This disclosure allows for overdriving an undersized coil, i.e. a coil rated for 10 Volts continuous, and briefly exceeds the continuous rating by applying e.g., 24 Volts. The effect of overdriving the coil is forces of a larger 24 Volt continuous coil are generated briefly during the pull-in phase, the period a solenoid valve's required forces are highest. During the one second pull-in, for example, the generated thermal power can be 240% of the allowable continuous power. This excess power is absorbed and accommodated by intentional design of the coil's thermal mass and implementing a low power third voltage state, during which, coil cooling occurs.

Expressed more simply, systems and methods herein can employ a solenoid coil with an undersized power rating at the nominal system voltage, i.e. an 11 Watt rating in a 24 Volt system. When driving at full system voltage, 24 Volts, the coil is briefly overdriven at 63 Watts, generating substantially more force. Those skilled in the art will readily appreciate that time spans, wattages, and voltages given herein as examples can instead be any suitable time span, wattage, or voltage for a given application.

The methods and systems of the present disclosure, as described above and shown in the drawings, provide for actuation of valves such as in insufflators, wherein the solenoids actuating the valves have improved size, weight, and efficiency than more traditional solenoid valves. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. A method comprising:
applying an actuating voltage to a solenoid for a first time span to actuate the solenoid against a bias; and
lowering voltage applied to the solenoid and to a holding voltage that is lower than the actuating voltage to hold the solenoid against the bias for a second span of time, wherein the solenoid has a coil to which the actuating and holding voltages are applied, wherein the coil is rated for continuous operation at a rated voltage, and wherein the actuating voltage is above the rated voltage.

2. The method as recited in claim 1, wherein the holding voltage is below the rated voltage.

3. The method as recited in claim 1, wherein applying the actuating voltage to the solenoid is performed after the bias is relaxed and voltage applied to the solenoid is dropped below the holding voltage for a span of time during which the coil has cooled to a first temperature, wherein the coil is raised to a second temperature during the second span of time.

4. The method as recited in claim 3, wherein the first time span is short enough so the coil does not exceed the second temperature during the first span of time.

5. The method as recited in claim 1, wherein the solenoid is operatively connected to a valve having an open position for allowing flow through the valve and a closed position for preventing flow through the valve, wherein the bias biases the valve to the open position, wherein actuating the solenoid against the bias actuates the valve to the closed position, and wherein holding the solenoid against the bias holds the valve in the closed position.

6. The method as recited in claim 5, further comprising lowering voltage below the holding voltage in response to over pressure in an a insufflation line, thereby opening the valve to the open position under the bias to relieve the over pressure in the insufflation line.

7. The method as recited in claim 5, further comprising lowering voltage to zero in a loss of power event, thereby opening the valve to the open position under the bias to relieve pressure in a surgical cavity in a patient.

8. The method as recited in claim 5, further comprising filtering flow through the valve in the open position to reduce or prevent particulates passing through the valve.

9. The method as recited in claim 1, wherein at least one of the actuating voltage and the holding voltage is applied as an alternating waveform that averages at the respective actuating and/or holding voltage.

10. The method as recited in claim 1, wherein the actuating voltage is higher than the rated voltage, but the first span of time is short enough to apply the first voltage without causing a failure mode for the coil wherein the failure mode includes at least one of insulation breakdown, housing or bobbin melting or distortion, or armature seizing.

11. The method as recited in claim 1, wherein the actuating voltage is 48 Volts and the holding voltage is 5 Volts.

12. The method as recited in claim 1, wherein applying the actuating voltage includes applying 20 Watts to the solenoid, and wherein holding the solenoid against the bias for a second span of time includes applying 1 Watt to the solenoid at the holding voltage.

13. A solenoid comprising:
a coil seated in a housing;
an armature extending in a longitudinal direction through the coil and out of a first end of the housing, wherein the armature includes a material responsive to external magnetic fields and is mounted in the hosing for movement relative to the coil and housing responsive to a magnetic field of the coil;
a latching member mounted at a second end of the housing and including a latching surface configured to contact the armature with the armature in a retracted position; and
a plurality of permanent magnets circumferentially distributed around the armature at the first end of the housing.

14. The solenoid as recited in claim 13, wherein the latching member is engaged with threads to the housing for adjusting axial position of the latching surface within the housing by turning the latching member relative to the housing.

15. The solenoid as recited in claim 13, wherein there are four permanent magnets circumferentially distributed about the armature.

16. The solenoid as recited in claim 15, wherein the polarity of each of the permanent magnets is oriented radially relative to the longitudinal direction.

17. The solenoid as recited in claim 16, wherein for each of the permanent magnets, a radially inner pole is magnetic south and a radially outer pole is magnetic north.

18. The solenoid as recited in claim 13, wherein the coil is wound around an outward surface of a bobbin of the housing, wherein an inward facing surface of the bobbin engages the armature as a sliding bearing surface.

19. A system comprising:
a solenoid including:
a coil seated in a housing; and
an armature extending in a longitudinal direction through the coil and out of a first end of the housing, wherein the armature includes a material responsive to external magnetic fields and is mounted in the hosing for movement relative to the coil and housing responsive to a magnetic field of the coil; and
a controller operatively connected to control the coil, wherein the controller includes machine readable instructions configured to cause the controller to:
apply an actuating voltage to the coil for a first time span to actuate the solenoid against a bias; and
lower voltage applied to the solenoid to a holding voltage that is lower than the actuating voltage to hold the solenoid against the bias for a second span of time, wherein the coil is rated for continuous operation at a rated voltage, and wherein the actuating voltage is above the rated voltage.

20. The system as recited in claim 19, further comprising:
a valve member mounted for movement together with the armature; and
a valve housing defining a flow path from an inlet of the valve housing to an outlet of the valve housing, wherein the flow path passes through a valve seat, wherein with the valve member and armature in a first position, the valve member seals against the valve seat preventing flow through the flow path, and with the valve member and armature in a second position, the valve member is spaced apart from the valve seat, allowing flow through the flow path.

21. The system as recited in claim 20, further comprising a filter medium seated in the flow path in the valve housing downstream of the valve seat, configured to prevent particle flow out of the valve housing with the armature and valve member in the second position.

22. The system as recited in claim 20, further comprising an insufflator, wherein the insufflator includes:
a pressure source; and
a pneumatic line connecting the pressure source to a connector configured to connect the insufflator to a trocar tube set for insufflating a surgical cavity of a patient, wherein the inlet of the valve housing is connected to a branch off of the pneumatic line to selectively allow or block pressure relief flow from the pneumatic line.
